(19) United States
(12) Patent Application Publication
(10) Pub. No.: US 2009/0118228 A1
(43) Pub. Date: May 7, 2009

(54) CAROTENOID-CONTAINING COMPOSITIONS AND METHODS

(75) Inventors: Zeina Jouni, Evansville, IN (US); Zeina Makhoul, Tucson, AZ (US)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(73) Assignee: Bristol-Myers Squibb Company

(21) Appl. No.: 12/206,850

(22) Filed: Sep. 9, 2008

Related U.S. Application Data
(60) Provisional application No. 60/986,102, filed on Nov. 7, 2007.

(51) Int. Cl. A61K 31/70; A61K 31/05; A61P 17/00; A61P 27/02; A61P 3/04; A61K 31/202

(52) U.S. Cl. 514/54; 514/733; 514/560

(57) ABSTRACT

The present invention is directed to methods for improving optical or skin health in a subject comprising administering to the subject a combination of lutein, zeaxanthin, lycopene, and beta-carotene.

### CROSS-REFERENCE TO RELATED PATENT APPLICATIONS

This application claims the priority benefit of U.S. Provisional Application No. 60/986,102, filed Nov. 7 2007, which is incorporated by reference herein in its entirety.

### BACKGROUND OF THE INVENTION

(1) Field of the Invention

The present invention relates generally to methods for improving optical and skin health via the administration of particular carotenoids.

(2) Description of the Related Art

A recent National Institutes of Health (NIH) study found that about 14 million Americans are visually impaired. Susan Vitale, et al., Prevalence of Visual Impairment in the United States, JAMA 295:2158-2163 (2006). Of these, more than 11 million have a visual impairment, such as nearsightedness, that can be corrected with glasses or contact lenses. The study confirms that uncorrected visual impairment is a major public health problem.

Eyesight is generally assessed by visual acuity testing. Visual acuity refers to the ability to distinguish closely adjacent components of a visual target, such as the separate strokes that make up a letter, and is a measure of how well a person sees. Someone with 20/20 visual acuity is just able to decipher a letter that contains component strokes separated by 1 minute of arc. A person with 20/40 visual acuity would need an object to be twice as large in order to decipher it at the same viewing distance as a person with 20/20 vision.

A person's visual acuity may be underdeveloped or may become impaired due to a variety of factors such as diseases, disorders, stresses, genetics or age. Impairments can range from blurred vision to complete blindness. Detecting and correcting visual acuity problems are important in adults and can be especially important in children and infants. Optimizing visual maturation during infancy or childhood may improve visual acuity throughout life. Therefore, it would be beneficial to provide a nutritional support regimen for infants that can support and improve visual acuity and optical health during childhood and throughout life.

In addition to a recent focus on optical health, focus on sun damage and its relationship to skin cancer has brought skin care and skin health to the forefront of health discussions. It is well accepted that skin cancer is the most common cancer in the US. In fact, statistics now show that one in five Americans will contract skin cancer during the course of their lifetime. We know that more than 90% of all skin cancers are caused by sun exposure, yet fewer than 33% of adults, adolescents, and children routinely use sun protection.

Protection against sun damage is especially critical in childhood and adolescence. More than half of a person's lifetime sun exposure occurs before the age of 20. The skin cancers that affect adults are partially a result of the sun damage they received in childhood and adolescence. For example, one blistering sunburn in childhood more than doubles the chances of developing melanoma later in life. Studies have shown that regular sun protection throughout childhood can reduce the risk of skin cancer by 80%. Therefore, it would also be beneficial to provide a nutritional support regimen for infants that can protect against sun damage and improve skin health throughout life.

### SUMMARY OF THE INVENTION

Briefly, therefore, the present invention is directed to a method for improving optical health in a subject comprising administering to the subject a combination of lutein, zeaxanthin, lycopene, and beta-carotene.

The invention is also directed to a method for preventing photoreceptor cell death in a subject comprising administering to the subject a combination of lutein, zeaxanthin, lycopene, and beta-carotene.

In addition, the invention is directed to a method for protecting a subject's eyes from light and sun damage comprising administering to the subject a combination of lutein, zeaxanthin, lycopene, and beta-carotene.

In other embodiments, the invention is directed to a method for improving skin health comprising administering to the subject a combination of lutein, zeaxanthin, lycopene, and beta-carotene.

In still other embodiments, the invention is directed to a method for preventing obesity in a subject comprising administering to the subject a combination of lutein, zeaxanthin, lycopene, and beta-carotene.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

Carotenoids are a related group of greater than 600 natural compounds, irrespective of geometric and stereoisomers, with demonstrated antioxidant efficacy. The carotenoids are broadly divided into “carotenes,” or non-oxygen substituted hydrocarbon carotenoids, and “xanthophylls,” oxygen-substituted carotenoids. Between 500 and 600 carotenoids have been identified, of which only about 24 occur in human foodstuffs. The major carotenoids found in foods are α-carotene, β-carotene, lycopene, lutein, zeaxanthin, and β-cryptoxanthin. They are present in foods such as carrots, pumpkins, sweet potatoes, tomatoes, and other deep green, yellow, orange, red fruits and vegetables. Most carotenoids occur in nature predominantly in the all-trans form. Three of these carotenoids, α-carotene, β-carotene and β-cryptoxanthin, can be converted into retinol and are therefore considered provitamin A carotenoids. Lycopene, lutein and zeaxanthin do not have a vitamin A function and are referred to as nonprovitamin A carotenoids.

An important feature of carotenoids is a centrally located, extended conjugated double-bond system, which is responsible for the chemical reactivity, light-absorbing properties, and, thus, color of carotenoids. Potential biological function is determined by the chemical structure of carotenoids. The alternating single and double bond of the polyene backbone of carotenoids allow them to absorb excess energy from other molecules, which accounts for their antioxidant properties. They perform their antioxidant function by either quenching singlet oxygen and/or blocking free radical-mediated reactions. The polarity of the specific end groups of carotenoids accounts for the differences in how they interact with biological membranes. Carotenoids are associated with lipid portions of human tissues, cells, and membranes and bind to hydrophobic surfaces because they are lipophilic. In addition, carotenoids are easily isomerized and oxidized due to their high unsaturation and hence may lose biological activity after processing and storage.

In recent years, carotenoids have received the interest of researchers from diverse fields including food science, pharmacy, biochemistry and nutrition because of their wide spectrum of biological functions such as provitamin A, antioxidant, immuno-enhancement, and prevention of degenerative diseases.

For example, see U.S. Pat. No. 6,579,544 to Rosenberg, et al., U.S. Pat. No. 6,573,299 to Petrus, et al., or U.S. Patent App. Pub. No, 2007/0166354 to Barrett-Reis, et al.

The technical problem to be solved by the present invention is to provide novel carotenoid nutritional compositions that are useful in improving optical and skin health in a subject. Thus, in an embodiment, the present invention is directed to methods for increasing macular optical density and/or preventing photoreceptor cell death in a subject in a subject by administering to them an effective amount of lutein, zeaxanthin, lycopene, and beta-carotene. The invention is also directed, in an embodiment, to a method for improving skin health via administration of lutein, zeaxanthin, lycopene, and beta-carotene.

Lutein is a carotenoid found in green leafy vegetables such as spinach and kale. The xanthophyll has primarily been used as a natural colorant due to its orange-red color. It absorbs blue light and, therefore, appears yellow at low concentrations and orange-red at high concentrations. Lutein is a lipophilic molecule and is generally insoluble in water. The presence of the long chromophore of conjugated double bonds (polyene chain) provides its distinctive light-absorbing properties. The polyene chain is susceptible to oxidative degradation by light or heat and is chemically unstable in acids.

The structural formula of lutein is as follows:

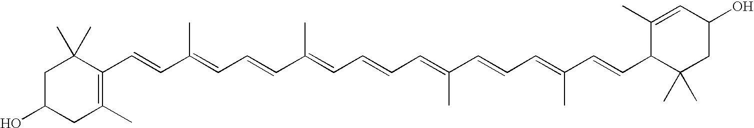

In an embodiment of the invention, the effective amount of lutein is within the range of about 0.01 mg and about 20 mg per kg body weight per day. In another embodiment of the invention, the effective amount of lutein is within the range of about 0.1 mg and about 10 mg per kg body weight per day. In a particular embodiment of the invention, the effective amount of lutein is about 6 mg per kg body weight per day.

Lutein and zeaxanthin have identical chemical formulas and are isomers, but they are not stereoisomers. The main difference between them is in the location of a double bond in one of the end rings. This difference gives lutein three chiral centers whereas zeaxanthin has two. The structural formula of zeaxanthin is as follows:

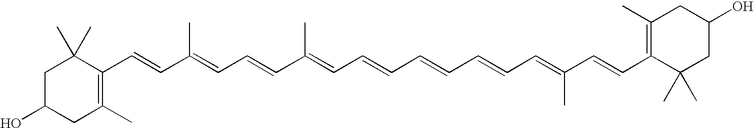

Zeaxanthin is one of the most common carotenoid alcohols found in nature. It is the pigment that gives corn, saffron, and many other plants their characteristic color. Zeaxanthin breaks down to form picrocrocin and safranal, which are responsible for the taste and aroma of saffron.

In an embodiment of the invention, the effective amount of zeaxanthin is within the range of about 0.01 mg and about 20 mg per kg body weight per day. In another embodiment of the invention, the effective amount of zeaxanthin is within the range of about 0.1 mg and about 10 mg per kg body weight per day. In a particular embodiment of the invention, the effective amount of zeaxanthin is about 6 mg per kg body weight per day.

Lycopene, similar to other carotenoids, is a natural fat-soluble red pigment and photochemical found in certain plants such as tomatoes, watermelon, papaya, pink grapefruit and pink guava. Lycopene may protect humans against certain disorders, such as cancer and coronary heart disease.

Lycopene is an acyclic isomer of beta-carotene. Lycopene is a 40 carbon atom, open chain polyisoprenoid with 11 conjugated double bonds. Lycopene is a terpene assembled from 8 isoprene units. The color of lycopene is due to its many conjugated carbon double bonds. Each double bond reduces the energy required for electrons to transition to higher energy states, allowing the molecule to absorb visible light of progressively longer wavelengths. Lycopene absorbs most of the visible spectrum, so it appears red.

The structural formula of lycopene is as follows:

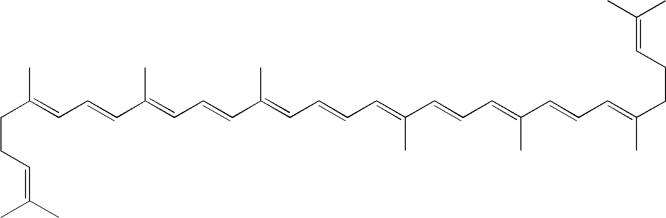

In an embodiment of the invention, the effective amount of lycopene is within the range of about 0.01 mg and about 10 mg per kg body weight per day. In another embodiment of the invention, the effective amount of lycopene is within the range of about 0.1 mg and about 5 mg per kg body weight per day. In a particular embodiment of the invention, the effective amount of lycopene is about 1 mg per kg body weight per day.

Like lycopene, beta-carotene is a carotenoid. Beta-carotene is the most common of the carotenes and can be found in yellow, orange, and green leafy fruits and vegetables. It is unclear whether beta-carotene has any biological function for humans other than as a precursor for vitamin A. There is some evidence that beta-carotene may play a beneficial role in human nutrition beyond its provitamin A function. Beta-carotene has antioxidant activity, at least in vitro, and it may enhance intercellular communication and may have immunomodulatory and anticarcinogenic activities in certain circumstances.

The structure of beta-carotene is set forth below:

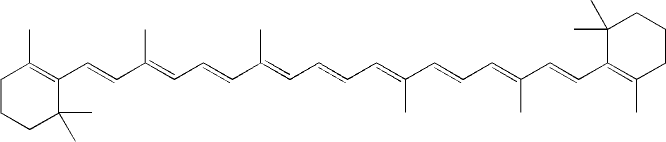

In an embodiment of the invention, the effective amount of beta-carotene is within the range of about 0.01 mg and about 10 mg per kg body weight per day. In another embodiment of the invention, the effective amount of beta-carotene is within the range of about 0.1 mg and about 5 mg per kg body weight per day. In a particular embodiment of the invention, the effective amount of beta-carotene is about 1 mg per kg body weight per day.

As noted, the present invention is directed to a method for optical and skin health in subjects by administering to them an effective amount of lutein, zeaxanthin, lycopene, and beta-carotene. Some of the optical benefits encompassed by the present invention include increasing macular optical density, increasing retinal pigment epithelial cell density, improving visual acuity, preventing photoreceptor cell death, providing protection from light and sun damage by filtering blue light. In addition, improvements in visual acuity may result in improvements in mental development.

Some of the skin benefits encompassed by the present invention include reduction of the formation of erythema, rash, and other skin problems that result from inflammation, infection, light and/or sun exposure. The present invention also encompasses an improvement in the skin through the ability of the formulation to absorb blue light. This may be particularly important for infants, as it is well established that their eyes and skin are very vulnerable to damage from blue light.

As used in the present invention, the source of the lutein, zeaxanthin, lycopene, and beta-carotene can be any source known in the art such as plant material, seafood, and/or single cell. In certain embodiments, one or more of the carotenoids may be in raw form or may be chemically manipulated. In a particular embodiment, one or more of the carotenoids may be genetically modified organisms.

In an embodiment, the lutein, zeaxanthin, lycopene, and beta-carotene may be administered in the form of a nutritional composition, infant formula, human milk supplement, or children's nutritional product. As used herein, the term “infant formula” means a composition that satisfies the nutrient requirements of an infant by being a substitute for human milk. Thus, the method of the invention is useful in preventing or treating bacterial infections in human infants, children, or adults.

If the lutein, zeaxanthin, lycopene, and beta-carotene are administered via an infant formula, the infant formula may be nutritionally complete and contain suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, and/or amino acids. Carbohydrate sources can be any used in the art, e.g., lactose, glucose, corn syrup solids, maltodextrins, sucrose, starch, and/or rice syrup solids. Lipid sources can be any used in the art, e.g., vegetable oils such as palm oil, canola oil, corn oil, soybean oil, palmolein, coconut oil, medium chain triglyceride oil, high oleic sunflower oil, and/or high oleic safflower oil.

Conveniently, commercially available nutritional compositions, infant formulas, human milk supplements, or children's nutritional products can be used. For example, Enfalac, Enfamil®, Enfamil® Premature Formula, Enfamil® with Iron, Enfamil® LIPIL®, Lactofree®, Nutramigen®, Pregestimil®, and ProSobee® (available from Mead Johnson & Company, Evansville, Ind., U.S.A.) may be supplemented with suitable levels of lutein, zeaxanthin, lycopene, and beta-carotene and used in practice of the method of the invention.

If the lutein, zeaxanthin, lycopene, and beta-carotene are administered in an infant formula, the amounts of each carotenoid in the formula may be up to about 40 nmol/g fat. In another embodiment, the amounts of each carotenoid in the formula may be within the range of about 2 nmol/g and about 35 nmol/g fat. In a particular embodiment, the amounts of each carotenoid in the formula may be within the range of about 5 nmol/g and about 30 nmol/g fat.

In other embodiments, if the lutein, zeaxanthin, lycopene, and beta-carotene are administered in an infant formula, the amounts of each carotenoid in the formula may be within the range of about 0.01 ppm and about 20 ppm carotenoid by weight of the total lipid content. In another embodiment, the amounts of each carotenoid in the formula may be within the range of about 0.1 ppm and about 10 ppm carotenoid by weight of the total lipid content.

The total carotenoid blend may comprise, in an embodiment, up to about 2000 mcg/L infant formula. In other embodiments, the total carotenoid blend may comprise from about 100 mcg/L to about 1500 mcg/L infant formula. In yet another embodiment, the total carotenoid blend may comprise from about 200 mcg/L to about 1200 mcg/L infant formula.

The individual carotenoids may be present in the infant formula in an amount of from about 50 mcg/L to about 1150 mcg/L, about 75 mcg/L to about 230 mcg/L, or about 100 mcg/L to about 200 mcg/L.

In some embodiments of the invention, additional components may be administered in combination with lutein, zeaxanthin, lycopene, and beta-carotene. These additional components may include probiotics, prebiotics, or long chain polyunsaturated fatty acids (LCPUFAs). The components may be administered separately from the lutein, zeaxanthin, lycopene, and beta-carotene or may be included as part of a nutritional composition, infant formula, human milk supplement, or children's nutritional product that contains lutein, zeaxanthin, lycopene, and beta-carotene, and one or more additional components.

The term “probiotic” means a microorganism that exerts beneficial effects on the health of the host. Any probiotic known in the art may be used, provided it is suitable for combination with the other components of the supplement. For example, the probiotic may be chosen from the group consisting of Lactobacillus and Bifidobacterium. Alternatively, the probiotic can be Lactobacillus rhamnosus GG.

The term “prebiotic”, as used herein, means a non-digestible food ingredient that stimulates the growth and/or activity of probiotics. In this embodiment, any prebiotic known in the art may be used, provided it is suitable for combination with the other components of the supplement. In a particular embodiment, the prebiotic can be selected from the group consisting of fructo-oligosaccharide, gluco-oligosaccharide, galacto-oligosaccharide, inulin, isomalto-oligosaccharide, polydextrose, xylo-oligosaccharide, lactulose, and combinations thereof. In a particular embodiment, the prebiobic is a mixture of galacto-oligosaccharide and polydextrose.

In an embodiment, the total amount of prebiotics present in the nutritional composition may be from about 1.0 g/L to about 10.0 g/L of the composition. In another embodiment, the total amount of prebiotics present in the nutritional composition may be from about 2.0 g/L and about 8.0 g/L of the composition. In yet another embodiment, the total amount of prebiotics present in the nutritional composition may be about 4.0 g/L of the composition.

If galacto-oligosaccharide is used as a prebiotic, the amount of galacto-oligosaccharide in the nutritional composition may, in an embodiment, be within the range of from about 1.0 g/L to about 4.0 g/L. In another embodiment, the amount of galacto-oligosaccharide in the nutritional composition may be about 2.0 g/L. If polydextrose is used as a prebiotic, the amount of polydextrose in the nutritional composition may, in an embodiment, be within the range of from about 1.0 g/L to about 4.0 g/L. In another embodiment, the amount of polydextrose in the nutritional composition may be about 2.0 g/L. In a particular embodiment, galacto-oligosaccharide and polydextrose are supplemented into the nutritional composition in a total amount of about 4.0 g/L. In this embodiment, the amount of galacto-oligosaccharide may be about 2.0 g/L and the amount of polydextrose may be about 2.0 g/L.

In yet another embodiment of the invention, LCPUFAs may be administered in combination with lutein, zeaxanthin, lycopene, and beta-carotene. In this embodiment, the LCPUFAs may include docosahexaenoic acid (DHA), arachidonic acid (ARA), eicosapentaenoic acid (EPA), and/or combinations thereof.

If administered as part of the present invention, the weight ratio of ARA:DHA may be from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

If administered as part of the present invention, the level of DHA may be within the range of about 0.0% and about 1.00% of fatty acids, by weight. In other embodiments, the level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

If administered as part of the present invention, the level of ARA may be within the range of about 0.0% and about 0.67% of fatty acids, by weight. In another embodiment, the level of ARA may be about 0.67% by weight. In another embodiment, the level of ARA may be about 0.5% by weight. In yet another embodiment, the level of DHA may be within the range of about 0.47% and about 0.48% by weight.

If administered as part of the present invention, the amount of DHA may be from about 2 mg/100 kilocalories (kcal) to about 100 mg/100 kcal. In another embodiment, the amount of DHA may be from about 5 mg/100 kcal to about 75 mg/100 kcal. In yet another embodiment, the amount of DHA may be from about 15 mg/100 kcal to about 60 mg/100 kcal.

If administered as part of the present invention, the amount of ARA may be from about 4 mg/100 kilocalories (kcal) to about 100 mg/100 kcal. In another embodiment, the amount of ARA may be from about 10 mg/100 kcal to about 67 mg/100 kcal. In yet another embodiment, the amount of ARA may be from about 20 mg/100 kcal to about 50 mg/100 kcal. In a particular embodiment, the amount of ARA may be from about 25 mg/100 kcal to about 40 mg/100 kcal. In one embodiment, the amount of ARA is about 30 mg/100 kcal.

If administered as part of the present invention, the effective amount of DHA may be from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

If administered as part of the present invention, the effective amount of ARA may be from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

If the composition of the invention is supplemented with oils containing LCPUFAs, it may be accomplished using standard techniques known in the art. For example, an equivalent amount of an oil which is normally present in a composition, such as high oleic sunflower oil, may be replaced with the LCPUFAs.

If utilized, the source of the LCPUFAs can be any source known in the art such as marine oil, fish oil, single cell oil, egg yolk lipid, and/or brain lipid. The LCPUFAs can be in natural form or refined form.

While not wishing to be bound to this or any theory, it is believed that the administration of LCPUFAs and carotenoids in the present invention may provide a synergistic effect. Any combination of DHA, ARA, EPA, and/or combinations thereof may provide a synergistic effect when administered in combination with lutein, zeaxanthin, lycopene, and beta-carotene according to the present invention. In some embodiments, this synergistic effect is seen through a combination of lutein, zeaxanthin, lycopene, beta-carotene, and DHA. In other embodiments, the synergistic effect is seen through a combination of lutein, zeaxanthin, lycopene, beta-carotene, DHA, and ARA. More specifically, it is believed that the administration of a combination of LCPUFAs and carotenoids, as described herein, may synergistically improve visual acuity, mental development, and mental performance in subjects.

In other embodiments of the invention, lutein, zeaxanthin, lycopene, and beta-carotene may be combined and administered to a subject for the purpose of treating or preventing any of the following. reflux, spitting up, abdominal pain, bloating, vomiting, gastric inflammation, gastritis, ulcer formation, hypertension, dyslipidemia, Type I and II diabetes, insulin sensitivity, obesity, cardiovascular disease, cancer, atherosclerosis. In other embodiments, lutein, zeaxanthin, lycopene, and beta-carotene can be combined and administered for the purpose of improving digestion or stool consistency, modulating antioxidant enzymes, decreasing cellular and tissue oxidative stress, shifting T-helper cell Types 1 to Th2 balance, and modulating immune function.

In some embodiments, the invention includes a method for improving weight management in a subject comprising administering to the subject an effective amount of lutein, zeaxanthin, lycopene, and beta-carotene. In other embodiments, the invention includes a method for preventing or treating obesity in a subject comprising administering to the subject an effective amount of lutein, zeaxanthin, lycopene, and beta-carotene. Obesity has been linked with an inflammation of adipose tissue. In some studies, inflammation has also been identified as an early characteristic of obesity. The combination of lutein, zeaxanthin, lycopene, and beta-carotene, in addition to their antioxidant benefits, may contribute to a reduction in inflammation, thereby reducing or preventing the onset of obesity in the present invention.

In an embodiment, the invention is directed to the use of a combination of lutein, zeaxanthin, lycopene, and beta-carotene in the manufacture of an ingestible composition for increasing macular optical density in a subject. In another embodiment, the invention is directed to the use of a combination of lutein, zeaxanthin, Iycopene, and beta-carotene in the manufacture of an ingestible composition for increasing retinal pigment epithelial cell density in a subject. In yet another embodiment, the invention is directed to the use of a combination of lutein, zeaxanthin, lycopene, and beta-carotene in the manufacture of an ingestible composition for preventing photoreceptor cell death in a subject. In still another embodiment, the invention is directed to the use of a combination of lutein, zeaxanthin, lycopene, and beta-carotene in the manufacture of an ingestible composition for improving skin health in a subject. In a further embodiment, the invention is directed to the use of a combination of lutein, zeaxanthin, lycopene, and beta-carotene in the manufacture of an ingestible composition for preventing obesity in a subject.

The invention is also directed, in an embodiment, to a combination of lutein, zeaxanthin, lycopene, and beta-carotene for use in increasing macular optical density in a subject. The invention is further directed, in an embodiment, to a combination of lutein, zeaxanthin, lycopene, and beta-carotene for use in increasing retinal pigment epithelial cell density in a subject. The invention is directed, in another embodiment, to a combination of lutein, zeaxanthin, lycopene, and beta-carotene for use in preventing photoreceptor cell death in a subject. The invention is additionally directed, in an embodiment, to a combination of lutein, zeaxanthin, lycopene, and beta-carotene for use in improving skin health in a subject. Further, the invention is directed, in an embodiment, to a combination of lutein, zeaxanthin, lycopene, and beta-carotene for use in preventing obesity in a subject.

All references cited in this specification, including without limitation, all papers, publications, patents, patent applications, presentations, texts, reports, manuscripts, brochures, books, internet postings, journal articles, and/or periodicals are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What is claimed is:

1. A method for increasing macular optical density in a subject comprising administering to the subject a combination of lutein, zeaxanthin, lycopene, and beta-carotene.

2. The method of claim 1 wherein the amount of lutein administered is within the range of about 0.01 mg and about 20 mg per kg body weight per day.

3. The method of claim 1 wherein the amount of lutein administered is within the range of about 0.1 mg and about 10 mg per kg body weight per day.

4. The method of claim 1 wherein the amount of zeaxanthin administered is within the range of about 0.01 mg and about 20 mg per kg body weight per day.

5. The method of claim 1 wherein the amount of zeaxanthin administered is within the range of about 0.1 mg and about 10 mg per kg body weight per day.

6. The method of claim 1 wherein the amount of lycopene administered is within the range of about 0.01 mg and about 10 mg per kg body weight per day.

7. The method of claim 1 wherein the amount of lycopene administered is within the range of about 0.1 mg and about 5 mg per kg body weight per day.

8. The method of claim 1 wherein the amount of beta-carotene administered is within the range of about 0.01 mg and about 10 mg per kg body weight per day.

9. The method of claim 1 wherein the amount of beta-carotene administered is within the range of about 0.1 mg and about 5 mg per kg body weight per day.

10. The method of claim 1 wherein the amount of lutein administered is within the range of about 0.1 mg and about 10 mg per kg body weight per day, the amount of zeaxanthin administered is within the range of about 0.1 mg and about 10 mg per kg body weight per day, the amount of lycopene administered is within the range of about 0.1 mg and about 5 mg per kg body weight per day, and the amount of beta-carotene administered is within the range of about 0.1 mg and about 5 mg per kg body weight per day.

11. The method of claim 1 additionally comprising the administration of at least one LCPUFA.

12. The method of claim 10 wherein the LCPUFA is selected from the group consisting of DHA, ARA, EPA, and combinations thereof.

13. The method of claim 1 additionally comprising the administration of at least one prebiotic.

14. The method of claim 13 wherein the prebiotic comprises a combination of galacto-oligosaccharide and polydextrose.

15 The method of claim 1 wherein the subject is an infant.

16. A method for increasing retinal pigment epithelial cell density in a subject comprising administering to the subject a combination of lutein, zeaxanthin, lycopene, and beta-carotene.

17. A method for preventing photoreceptor cell death in a subject comprising administering to the subject a combination of lutein, zeaxanthin, lycopene, and beta-carotene.

18. A method for improving skin health in a subject comprising administering to the subject a combination of lutein, zeaxanthin, lycopene, and beta-carotene.

19. The method of claim 18 wherein the improvement in skin health comprises a reduction of erythema or rash.

20. A method for preventing obesity in a subject comprising administering to the subject a combination of lutein, zeaxanthin, lycopene, and beta-carotene.
